(19) United States
(12) Patent Application Publication
(10) Pub. No.: US 2020/0054587 A1
(43) Pub. Date: Feb. 20, 2020

(54) METHODS AND MATERIALS FOR REDUCING SCARRING ASSOCIATED WITH ARTHROFIBROSIS OR TENDON INJURY

(71) Applicant: Mayo Foundation for Medical Education and Research, Rochester, MN (US)

(72) Inventors: Saranya P. Wyles, Rochester, MN (US); Cody C. Wyles, Rochester, MN (US); Alexander Meves, Rochester, MN (US); Steven L. Moran, Rochester, MN (US)

(73) Assignee: Mayo Foundation for Medical Education and Research, Rochester, MN (US)

(21) Appl. No.: 16/541,610

(22) Filed: Aug. 15, 2019

Related U.S. Application Data
(60) Provisional application No. 62/764,932, filed on Aug. 16, 2018.

(51) Int. Cl. A61K 31/155; A61P 21/00; A61K 9/00

(52) U.S. Cl. CPC A61K 31/155; A61K 9/0019; A61P 21/00

(57) ABSTRACT

This document provides methods and materials for reducing arthrofibrosis at a wound site and/or reducing scar formation and adhesion after tendon injury. For example, provided herein are methods and materials for administering pentamidine to reduce arthrofibrosis and/or scarring and adhesion formation associated with a wound or tendon injury.

### CROSS-REFERENCE TO RELATED APPLICATIONS

This application claims benefit of priority from U.S. Provisional Application Ser. No. 62/764,932, filed on Aug. 16, 2018. The disclosure of the prior application is considered part of (and is incorporated by reference in) the disclosure of this application.

### BACKGROUND

### 1. Technical Field

This document relates to methods and materials for reducing arthrofibrosis, and/or for reducing scarring and adhesion formation associated with tendon injury. For example, this document relates to methods and materials for administering pentamidine (4,4′-[pentane-1,5-diylbis(oxy)]dibenzenecarboximid-amide) to reduce arthrofibrosis and scar formation associated with injury to a tendon, such as the flexor tendon.

### 2. Background Information

Arthrofibrosis is the formation of excessive scar tissue around a joint after injury or surgery. It often is characterized by pain and stiffness of the joint. Clinically, patients present with a restricted range of motion (ROM) (Flexion 140°-143°). Arthrofibrosis is an inflammatory process mediated by mast cell proliferation and the release of cytokine signaling proteins from mast cells.

In the human hand, the flexor tendons attach muscles in the forearm to the bones in the fingers, allowing the fingers to bend when the forearm muscles contract. An injury to the forearm, fingers, thumb, wrist, or hand can damage the flexor tendons, causing pain and reducing the ability to move the hand and/or fingers. Injuries typically are usually caused by a deep cut or an athletic injury, but rheumatoid arthritis can also cause weakening and tearing of the flexor tendons.

### SUMMARY

This document provides methods and materials for reducing arthrofibrosis, and for reducing scarring and adhesion formation associated with tendon injury (e.g., flexor tendon injury, rotator cuff injury, and other tendon injuries). For example, this document provides methods and materials for intraarticularly administering pentamidine to reduce arthrofibrosis, and to reduce scar formation after tendon injury.

As described herein, a composition containing pentamidine (e.g., a composition containing pentamidine as the sole active ingredient or in combination with one or more other active ingredients) or a related compound (e.g., pentamidine isethionate) can be administered to a mammal (e.g., to the intraarticular joint space of a mammal, such as a human) to reduce arthrofibrosis and/or to reduce scar formation after tendon injury. For example, a composition containing pentamidine can be administered to a wound (e.g., an accidental cut or surgical incision in the area of a joint or a tendon) in a manner that results in healing with reduced scar formation as compared to the healing that occurs when a comparable wound heals without the administration of a composition containing pentamidine. Thus, the use of pentamidine-containing compositions as provided herein can allow a user (e.g., a human) to experience joint and tendon healing with reduced scar formation.

In a first aspect, this document features a method for healing a wound of a mammal with reduced arthrofibrosis. The method can include applying a composition containing pentamidine (or a salt thereof) or pentamidine isethionate (or a salt thereof) to the wound of the mammal, where the wound heals with less arthrofibrosis than a comparable wound healed in the absence of the composition. The mammal can be a human. The wound can be a surgical incision or an accidental wound. The composition can contain from about 0.01 percent to about 10 percent, by weight, of the pentamidine (or a salt thereof) or pentamidine isethionate (or a salt thereof). The composition can be applied directly to tissue within the wound while the wound is open, or can be injected into the intraarticular space after the wound is closed.

In another aspect this document features a method for healing a wound of a mammal with reduced scar formation, where the wound is associated with a tendon injury. The method can include applying a composition containing pentamidine (or a salt thereof) or pentamidine isethionate (or a salt thereof) to an injured tendon of the mammal, where the wound heals with less scar formation than a comparable wound healed in the absence of the composition. The mammal can be a human. The tendon can be a flexor tendon. The tendon injury can be a surgical injury or an accidental injury. The composition can contain from about 0.01 percent to about 10 percent, by weight, of the pentamidine (or a salt thereof) or pentamidine isethionate (or a salt thereof). The composition can be directly applied to the tendon and/or tissue in the vicinity of the tendon while the wound is open, or can be injected into the mammal in the vicinity of the tendon (e.g., into an intraarticular space) after the wound is closed.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1D show the results of cytotoxicity studies. FIGS. 1A and 1B are images showing adipose tissue-derived mesenchymal stem cells (aMSC) stained with Calcein AM to indicate live cells (FIG. 1A) or Ethidium Homdimer-1 to indicate dead cells (FIG. 1B) after 24 hours of exposure to 100 μM pentamidine. FIGS. 1C and 1D are graphs plotting the results of MTS (3-(4,5-dimethylthiazol-2-yl)-5-(3-carboxymethoxy-phenyl)-2-(4-sulfophenyl)-2H-tetrazolium) assays of aMSC treated with the indicated concentrations of pentamidine for 30 minutes (FIG. 1C) or 180 minutes (FIG. 1D).

FIG. 2 is a graph plotting the results of passive range of motion assessment for rabbits subjected to stimulated fibrosis and contracture as described herein, with or without treatment with pentamidine for the indicated lengths of time.

FIGS. 3A and 3B are a pair of graphs plotting expression of collagen genes (FIG. 3A) and expression of the alpha smooth muscle actin gene (FIG. 3B) in tissue from rabbits subjected to stimulated fibrosis and contracture as described herein, with or without pentamidine treatment.

FIG. 4 is a graph plotting adhesion scores for turkeys subjected to surgical flexor tendon cut and repair, and then treated or not treated with pentamidine isethionate for six weeks.

### DETAILED DESCRIPTION

Pentamidine is an antimicrobial agent that plays a role in the reduction of inflammatory cytokines through its effects on mast cells. The studies described herein demonstrate pentamidine's effect on reducing arthrofibrosis, as well as pentamidine's effect on reducing scar formation after flexor tendon injury. This document therefore provides methods and materials involved in treating a mammal to reduce arthrofibrosis at a wound site or scar formation after tendon injury by administering pentamidine (or a salt thereof) or pentamidine isethionate (or a salt thereof) to the mammal. Any appropriate subject can be treated as described herein. For example, humans and other primates such as monkeys can be treated with pentamidine (or a salt thereof, or pentamidine isethionate or a salt thereof) to reduce arthrofibrosis or scar formation associated with flexor tendon injury. In some cases, mammals such as dogs, cats, horses, bovine species, porcine species, mice, or rats, or fowl such as turkeys or chickens, can be treated with pentamidine (or a salt thereof, or pentamidine isethionate or a salt thereof) as described herein to reduce arthrofibrosis at a wound site or scar formation associated with tendon injury. Moreover, a subject having any type of wound can be treated as described herein. For example, a mammal having an accidental wound or a surgical incision can be treated with pentamidine (or a salt thereof, or pentamidine isethionate or a salt thereof) as described herein to reduce arthrofibrosis at a wound site or to reduce scar formation associated with tendon injury. In some cases, a mammal (e.g., a human) developing or expected to develop arthrofibrosis at a wound site or scar formation associated with flexor tendon injury can be treated with pentamidine (or a salt thereof, or pentamidine isethionate or a salt thereof) as described herein.

Once identified as having a wound or a susceptibility to develop arthrofibrosis or scarring after tendon injury, the mammal can be administered a composition containing pentamidine (or a salt thereof, or pentamidine isethionate or a salt thereof) to the affected areas of bone or tendon. In some cases, pentamidine (or a salt thereof, or pentamidine isethionate or a salt thereof) can be administered as the sole active ingredient. In other cases, pentamidine (or a salt thereof, or pentamidine isethionate or a salt thereof) can be administered in combination with, for example, one or more additional agents (e.g., a corticosteroid, niclosamide, 5-fluorouracil, a hydrogel scaffold, or combinations thereof). Examples of corticosteroids that can be used in combination with pentamidine include, without limitation, hydrocortisone and triamcinolone.

A composition containing pentamidine (or a salt thereof, or pentamidine isethionate or a salt thereof) can be administered to a subject (e.g., to a mammal's joint space or to tissue accessible via a surgical or accidental wound) once or more than once, for any appropriate period of time (e.g., days, weeks, or months). For example, a composition provided herein can be applied once at the time of a surgical procedure (e.g., one intraarticular application). In some cases, a composition containing pentamidine (or a salt thereof, or pentamidine isethionate or a salt thereof) can be repeatedly administered to a subject (e.g., a mammal, such as a human) until the wound or tendon being treated is healed. For example, a composition containing pentamidine (or a salt thereof, or pentamidine isethionate or a salt thereof) can be administered orally, topically, or by injection (e.g., by injecting a pentamidine gel into an intraarticular joint space of a mammal) one or more times a week (e.g., one, two, three, four, or five times a week) or one or more times a month (e.g., one, two, three, four, or five times a month) for at least one, two, three, or four weeks, or at least one, two, three, four, five, six, seven, or eight months. In some cases, pentamidine (or a salt thereof, or pentamidine isethionate or a salt thereof) can be administered as a solid (e.g., as a powder), which can be applied directly to a tendon, a joint, and/or adjacent tissue. Administration of pentamidine (or a salt thereof, or pentamidine isethionate or a salt thereof) as a solid can be carried at the time of a surgical procedure, for example.

A composition for use in the methods provided herein can contain any appropriate amount of pentamidine (or a salt thereof, or pentamidine isethionate or a salt thereof). For example, a composition provided herein can contain from about 0.01 percent to about 15 percent (e.g., from about 0.01 percent to about 0.1 percent, from about 0.01 percent to about 5 percent, from about 0.01 percent to about 10 percent, from about 0.05 percent to about 0.5 percent, from about 0.1 percent to about 0.5 percent, from about 0.1 percent to about 1 percent, from about 0.1 percent to about 10 percent, from about 0.1 percent to about 15 percent, from about 0.5 percent to about 1 percent, from about 1 percent to about 3 percent, from about 1 percent to about 5 percent, from about 1 percent to about 10 percent, or from about 1 percent to about 15 percent), by weight, of pentamidine (or a salt thereof, or pentamidine isethionate or a salt thereof). In some cases, a composition provided herein can include a fixed number of active ingredients. For example, a composition useful in the methods provided herein can be formulated to have no more than one active ingredient (i.e., pentamidine or a salt thereof, or pentamidine isethionate or a salt thereof), or no more than two, no more than three, or no more than four active ingredients. In some cases, a composition provided herein can be formulated to have two active ingredients (e.g., pentamidine and niclosamide) and no other active ingredients.

A composition that can be used in the methods provided herein can be in any appropriate form for intraarticular application to a joint space (e.g., a human joint) or to a bone or a tendon accessible within a wound (e.g., an injury or a surgical incision). For example, a composition used in the methods provided herein can be in the form of a cream, gel, spray (e.g., an aerosol spray or non-aerosol spray), ointment, lotion, foam, solution, paste, powder, or clay. In some cases, a pentamidine composition can be formulated for oral delivery.

In some cases, a composition provided herein can contain one or more optional classes of ingredients such pH adjusters, preservatives, solvents, viscosity increasing agents, excipients, emulsifiers, and emollients. In some cases, a composition provided herein can contain a topical silicone-based compounding base agent such as PRACASIL™-Plus.

The final pH of an undiluted composition for use in the methods provided herein can be between about 5 and about 8. To obtain such a final pH, the pH of the composition can be adjusted. A pH-adjusting agent can be used to adjust the pH. The pH adjustment can be accomplished with any of a wide variety of acids if the composition has a pH that is above the desired range (e.g., above 8 before adjustment). Examples of acids that can be used to lower the pH of such formulations include, without limitation, citric acid, acetic acid, benzoic acid, glycolic acid, lactic acid, malic acid, and sulfuric acid if the composition has a pH that is too high (e.g., greater than 8 before adjustment). Likewise, the pH adjustment can be accomplished with any of a wide variety of bases should the composition have a pH that is too low (e.g., less than 5 before adjustment). Non-limiting examples of bases that can be used to increase the pH of these formulations include potassium hydroxide, potassium carbonate, sodium carbonate, sodium hydroxide, ethanolamine, or triethanolamine.

In some cases, a preservative can be included in a composition described herein to preserve the composition and prevent microbial growth. Examples of preservatives that can be included in a composition include, without limitation, butylated hydroxytoluene, benzoic acid, benzyl alcohol, butylparaben, propylparaben, methyparaben, DMDM hydantoin, potassium benzoate, methylisothiazolinone, methylchloroisothiazolinone, phenoxyethanol, quaterium-8, quaterium-14, quaterium-15, triclosan, zinc pyrithione, and zinc salicylate.

In some cases, one or more solvents can be included in a composition provided herein as an optional ingredient. Examples of solvents that can be included in a composition provided herein include, without limitation, butanediol, isoparaffin, cyclomethicone, ethoxyglycol, glycerin, mineral oil, polydimethlysiloxanes, propylene glycol, and propanediol.

In some cases, to help acquire a desired finished product thickness or viscosity, one or more viscosity modifiers can be included in a composition provided herein. Examples of viscosity modifiers that can be included in a composition provided herein include, without limitation, zinc oxide, ammonium xylene sulfonate, bentonite, calcium alginate, cocamide DEA, cocamide MEA, dextrin, hectorite, ethylcellulose, guar hydroxypropyltrimonium chloride, hydroxypropyl guar, hydrated silica, lauramide DEA, lauramide MEA, magnesium chloride, methylcellulose, pectin, polyethyleneglycol (PEGs), sodium chloride, sodium stearate, xanthan gum, and corn starch.

In some cases, one or more excipients can be included in a composition provided herein. Examples of excipients that can be included in a composition provided herein include, without limitation, menthol, diglyceride, triglyceride, stabilizing agents, antioxidants, fragrances, and colorants.

In some cases, one or more emulsifiers and emollients can be included in a composition provided herein. Examples of emulsifiers and emollients that can be included in a composition provided herein include, without limitation, ceteareth-20, cetostearyl alcohol, diethylaminethyl stearate, glyceryl dilaurate, glyceryl monostearate, glyceryl stearate, PEG-100 stearate, octyldodecyl stearoyl stearate, polysorbate 80, quaternium-2β, stearyl alcohol, sodium PCA, dimethicone, cyclomethicone, propylene glycol, and polysiloxane derivatives. In some cases, an emulsifier or emollient can be present in a composition provided herein at an amount from about 20 percent to about 80 percent, by weight.

The invention will be further described in the following examples, which do not limit the scope of the invention described in the claims.

### EXAMPLES

### Example 1—Pentamidine Effects on Arthrofibrosis

Pentamidine isethionate (a diamidino compound) is used in the treatment of Pneumocystis carinii pneumonia, and has been primarily studied in aerosolized and oral form (Sands et al., Rev Infect Dis, 7:625, 1985; and Rosenthal et al., Toxicol Appl Pharmacol, 107:555, 1990). Pentamidine may inhibit the release of inflammatory mediators from alveolar macrophages, which may be associated with its anti-parasite activity. Topical pentamidine (at a minimum dose of 20 μg) also was studied in the inhibition of contact hypersensitivity reaction in Langerhans cells (Blaylock et al., J Immunol, 147(7):2116-2121, 1991).

The following studies were performed to investigate the use of pentamidine for reducing arthrofibrosis-related scarring, and to investigate the use of pentamidine for reducing scarring resulting from flexor tending injury.

PENTAM® 300 (pentamidine isethionate) is an anti-protozoal agent and is a sterile, nonpyrogenic, lyophilized product. Pentamidine isethionate is a white crystalline powder soluble in water and glycerin, soluble in alcohol, and insoluble in ether, acetone, and chloroform. It is chemically designated as 4,4-[1,5-pentanediylbis(oxy)]bis-benzenecarboximidamid. A biocompatible sterile gel composed of carbomer, triethanolamine, and monopropylene glycol was utilized to compound the active ingredient. Fifty (50) g of intraarticular pentamidine in the biocompatible sterile gel was produced for the initial animal studies in a USP-grade facility. This gel was stored at room temperature and was stable until the end of the study period (6 weeks)

A rabbit model described elsewhere (Reina et al., J Orthopaedic Res DOI: 10.1002/jor.23884) was used to study the effects of pentamidine on knee arthrofibrosis over a 24 week period. Twelve rabbits underwent surgery on the right knee to stimulate fibrosis and contracture, as seen following surgery or trauma. All rabbits underwent luxation (hyperextension) and arthrodesis (fixation) of the operative knee. Pentamidine gel (50 mg lyophilized powder in mixed in ultrasound gel) was injected into the joint capsule of six rabbits' operative limb, during surgery. After eight weeks, the arthrodesis was reversed. Rabbits were allowed to roam free in individual spaces (1 m²) for the entire 24 week duration. Angles of passive extension of operative and contralateral, non-operative limbs were measured at 8, 10, 16, and 24 weeks, using a validated dynamic load cell device. Rabbits were sacrificed at 24 weeks and samples of the posterior capsule were taking from the operative and contralateral, non-operative knee. Data from mRNA extraction and qPCR were obtained to analyze expression of alpha-1 collagen (COL1A1, COL2A1 and COL5A1) and alpha smooth muscle actin (ACTA2), all of which are involved in fibrosis.

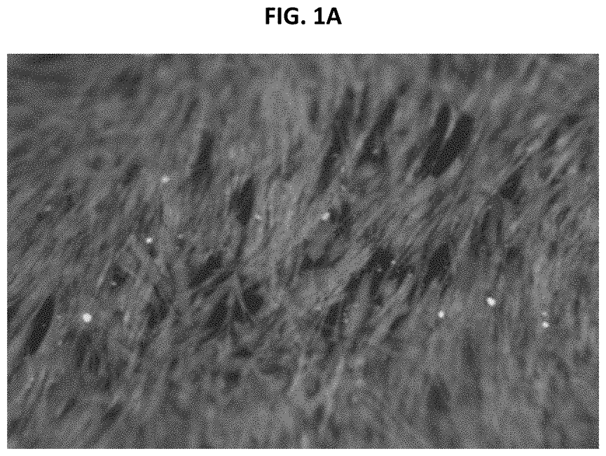

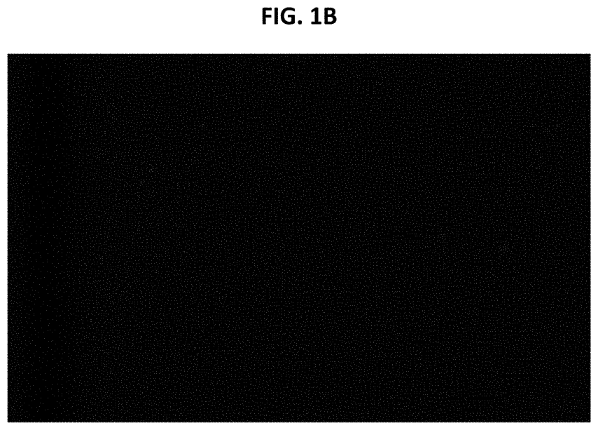

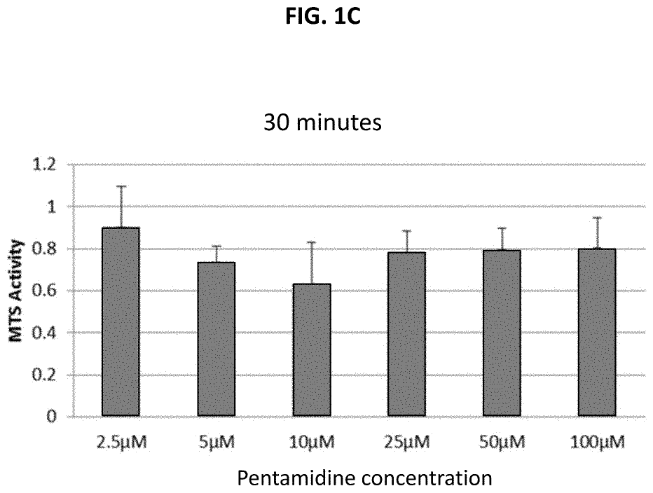

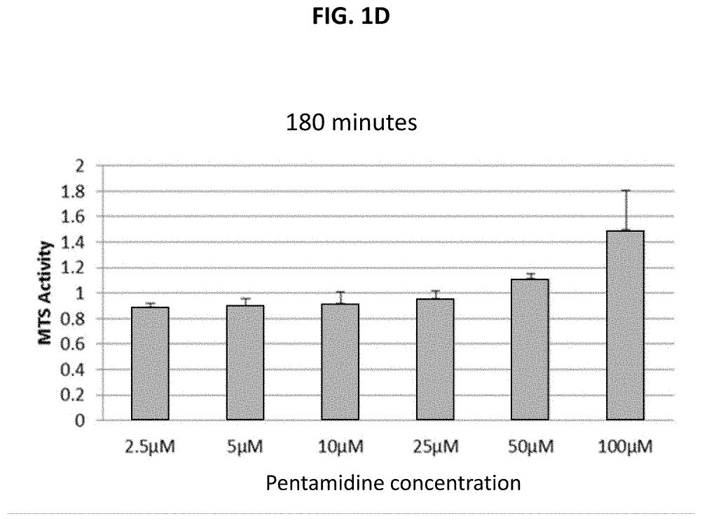

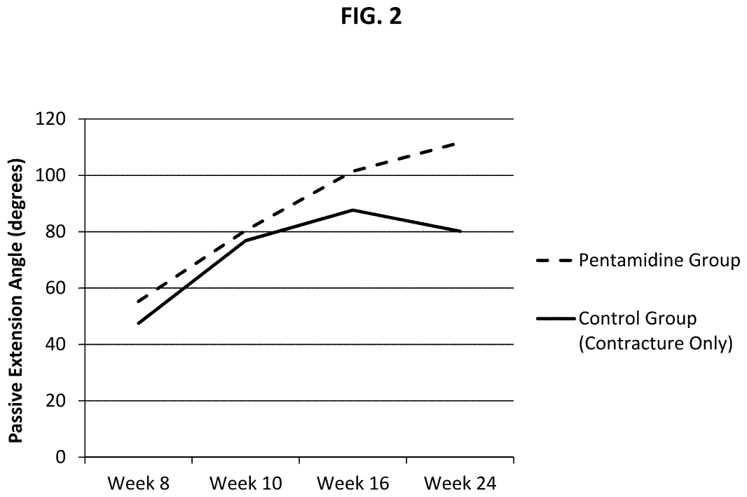

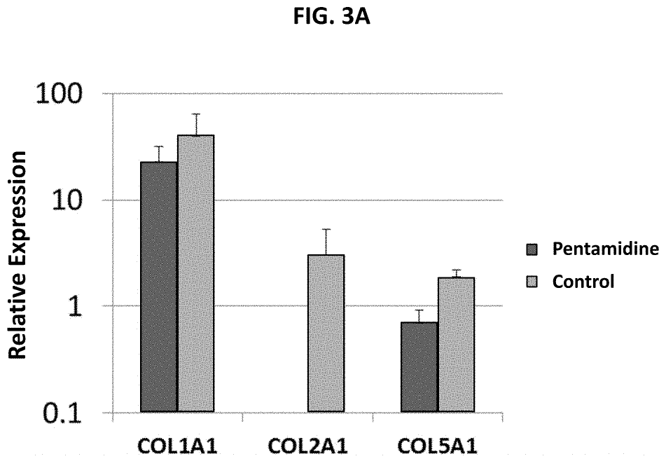

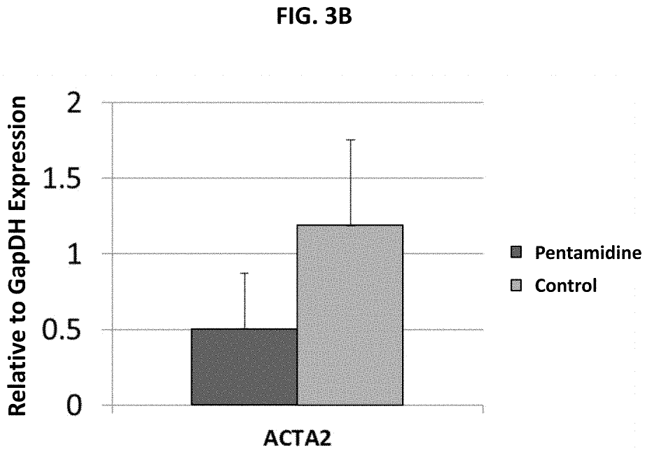

The cytotoxicity of pentamidine was assessed. Adipose tissue-derived mesenchymal stem cells (aMSC) survived after 24 hours of exposure to pentamidine solution (FIGS. 1A and 1B). The absence of any Ethidium Homdimer-1 staining (FIG. 1B) indicated that there was no cytotoxic effect after initial exposure. Various concentrations (2.5 μM to 100 μM pentamidine) were tested and validated to be safe. Exposure to pentamidine did not affect aMSC metabolism (FIGS. 1C and 1D). There was a statistically significant difference in the passive range of motion (ROM) between pentamidine and control rabbits at eight weeks (p=0.0303) and 24 weeks (p=0.0082) (FIG. 2), suggesting that pentamidine treatment improved passive extension angle and joint mobility. In addition, collagen (COL1A1, COL2A1 and COL5A1) expression was decreased in pentamidine-treated rabbit tissue as compared to control tissue (FIG. 3A), and ACTA2 gene expression also was decreased in pentamidine-treated rabbit tissue as compared to control tissue (FIG. 3B).

Thus, this study showed that pentamidine downregulated mast cell gene expression of known inflammatory markers, suggesting that pentamidine plays a role in anti-inflammatory effects on mast cells, and indicating that pentamidine is a promising and novel candidate for use in the treatment and prevention of arthrofibrosis after total knee arthroplasty and trauma.

### Example 2—Pentamidine Effects on Flexor Tendon Injuries

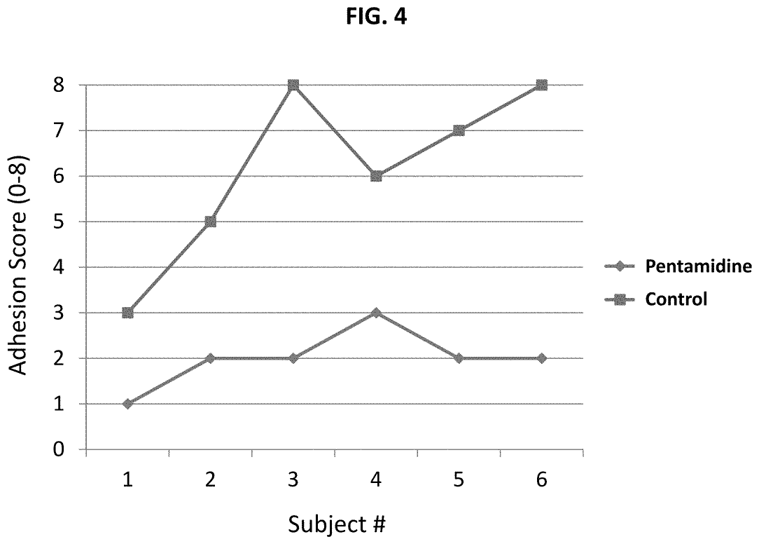

Flexor tendon injuries are common and pose a clinical challenge for functional restoration. Postoperative repair rupture and scar formation results in prolonged work disability for patients. The efficacy of pentamidine isethionate (50 mg lyophilized powder) was assessed in an in vivo turkey flexor tendon injury model (Kadar et al., J Surg Res 216:46-55, 2017). Twelve male turkeys underwent surgical flexor tendon cut and repair. Turkeys were postoperatively allocated to pentamidine-treated (n=6) control (untreated; n=6) groups, and were sacrificed after 6 weeks. For the treated group, pentamidine powder (50 mg) was applied to a repaired tendon after it had been cut, and surgical closure was performed per standard protocol. After sacrifice, digits were collected and analyzed for adhesion formation as well as healing at the macrolevel. All turkeys survived anesthesia and surgery. Gross anatomy revealed a marked reduction in post-operative scar formation in the pentamidine-treated group compared to control. Based on adhesion scores ranging from 0 (no adhesion) to 8 (very severe, >20 mm in length and cannot be separated), pentamidine treatment significantly reduced the adhesion score in all six treated turkeys (FIG. 4).

This study therefore demonstrated, for the first time, the effects of pentamidine on reducing post-operative scar formation in flexor tendon injury. These effects may occur through inhibition of osteopontin.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for healing a wound of a mammal with reduced arthrofibrosis, wherein said method comprises applying a composition comprising pentamidine or pentamidine isethionate to a wound of a mammal, wherein said wound heals with less arthrofibrosis than a comparable wound healed in the absence of said composition.

2. The method of claim 1, wherein said mammal is a human.

3. The method of claim 1, wherein said wound is a surgical incision.

4. The method of claim 1, wherein said wound is an accidental wound.

5. The method of claim 1, wherein said composition comprises from about 0.01 percent to about 10 percent, by weight, of said pentamidine or pentamidine isethionate.

6. The method of claim 1, wherein said composition is applied directly to tissue within said wound while said wound is open.

7. The method of claim 1, wherein said composition is injected into said mammal after said wound is closed.

8. A method for healing a wound of a mammal with reduced scar formation, wherein said wound is associated with tendon injury, wherein said method comprises applying a composition comprising pentamidine or pentamidine isethionate to an injured tendon of said mammal, and wherein said wound heals with less scar formation than a comparable wound healed in the absence of said composition.

9. The method of claim 8, wherein said mammal is a human.

10. The method of claim 8, wherein said tendon is a flexor tendon.

11. The method of claim 8, wherein said tendon injury is a surgical injury.

12. The method of claim 8, wherein said tendon injury is an accidental injury.

13. The method of claim 8, wherein said composition comprises from about 0.01 percent to about 10 percent, by weight, of said pentamidine or pentamidine isethionate.

14. The method of claim 8, wherein said composition is applied directly to said tendon and tissue in the vicinity of said tendon while said wound is open.

15. The method of claim 8, wherein said composition is injected into said mammal in the vicinity of said tendon after said wound is closed.
